(19) United States
(12) Patent Application Publication
(10) Pub. No.: US 2024/0122781 A1
(43) Pub. Date: Apr. 18, 2024

(54) INFORMATION PROCESSING DEVICE, INFORMATION PROCESSING METHOD, AND PROGRAM

(71) Applicant: SONY GROUP CORPORATION, TOKYO (JP)

(72) Inventors: MASAYUKI YOKOYAMA, TOKYO (JP); JUNYA SUZUKI, TOKYO (JP)

(21) Appl. No.: 18/264,148

(22) PCT Filed: Jan. 5, 2022
(86) PCT No.: PCT/JP2022/000065

§ 371 Date: Aug. 3, 2023

(30) Foreign Application Priority Data
Feb. 15, 2021 (JP) 2021-021489

(51) Int. Cl. A61H 3/06; A61F 9/08; G01S 15/08; G08B 3/10; H04S 7/00

(52) U.S. Cl. CPC A61H 3/061; A61F 9/08; G01S 15/08; G08B 3/1008; H04S 7/303; H04S 2400/13; H04S 2420/01

(57) ABSTRACT

The present technology relates to an information processing device, an information processing method, and a program that enable a user to perceive a surrounding situation reliably and stably. A distance and a direction of a measurement point with respect to a first position in a first coordinate system are calculated based on a relative distance and direction in the first coordinate system or a second coordinate system between the first position whose coordinates in the first coordinate system are determined and a second position whose coordinates in the second coordinate system are determined, the second position being separated from the first position, a relative attitude between the first coordinate system and the second coordinate system, and a distance to the measurement point present in a predetermined measurement direction in the second coordinate system, measured from the second position, and a notification signal to be presented to a user is generated based on at least one of the distance and the direction of the measurement point with respect to the first position. The present technology can be applied to an obstacle notification system that notifies a visually impaired person or the like of the presence of an obstacle.

### TECHNICAL FIELD

The present technology relates to an information processing device, an information processing method, and a program, and more particularly to an information processing device, an information processing method, and a program that enable a user to perceive a surrounding situation reliably and stably.

### BACKGROUND ART

PTL 1 and PTL 2 disclose a system in which a visually impaired person perceives the surrounding situation from echo sounds of actually emitted test sounds or from simulated echo sounds generated from the actually measured positions of objects.

### CITATION LIST

### Patent Literature

[PTL 1]
JP 2018-75178 A
[PTL 2]
JP 2018-78444 A

### SUMMARY

### Technical Problem

If the position of a sound emitting device that emits a test sound or the position of a sensor that actually measures the position of an object changes with respect to the position of the head (ear) of a visually impaired user, the user will be able to perceive the surrounding situation reliably and stably by hearing.

The present technology has been made in view of such circumstances, and enables the user to perceive the surrounding situation reliably and stably.

### Solution to Problem

An information processing device or a program according to the present technology is an information processing device or a program for causing a computer to function as the information processing device, the information processing device including a processing unit that calculates a distance and a direction of a measurement point with respect to a first position in a first coordinate system based on a relative distance and direction in the first coordinate system or a second coordinate system between the first position whose coordinates in the first coordinate system are determined and a second position whose coordinates in the second coordinate system are determined, the second position being separated from the first position, a relative attitude between the first coordinate system and the second coordinate system, and a distance to the measurement point present in a predetermined measurement direction in the second coordinate system, measured from the second position, and generates a notification signal to be presented to a user based on at least one of the distance and the direction of the measurement point with respect to the first position.

An information processing method of the present technology is an information processing method for causing a processing unit of an information processing device to execute: calculating a distance and a direction of a measurement point with respect to a first position in a first coordinate system based on a relative distance and direction in the first coordinate system or a second coordinate system between the first position whose coordinates in the first coordinate system are determined and a second position whose coordinates in the second coordinate system are determined, the second position being separated from the first position, a relative attitude between the first coordinate system and the second coordinate system, and a distance to the measurement point present in a predetermined measurement direction in the second coordinate system, measured from the second position, and generating a notification signal to be presented to a user based on at least one of the distance and the direction of the measurement point with respect to the first position.

In the information processing device, the information processing method, and the program of the present technology, a distance and a direction of a measurement point with respect to a first position in a first coordinate system are calculated based on a relative distance and direction in the first coordinate system or a second coordinate system between the first position whose coordinates in the first coordinate system are determined and a second position whose coordinates in the second coordinate system are determined, the second position being separated from the first position, a relative attitude between the first coordinate system and the second coordinate system, and a distance to the measurement point present in a predetermined measurement direction in the second coordinate system, measured from the second position, and a notification signal to be presented to a user is generated based on at least one of the distance and the direction of the measurement point with respect to the first position.

### BRIEF DESCRIPTION OF DRAWINGS

FIG. 1 is a configuration diagram showing a configuration example of an embodiment of an obstacle notification system to which the present technology is applied.

FIG. 2 is a diagram explaining the principle of measuring the three-dimensional position of an obstacle (the measurement point) in the obstacle notification system in FIG. 1.

FIG. 3 is a flowchart illustrating a processing procedure of the obstacle notification system in FIG. 1.

FIG. 4 is a block diagram illustrating the internal configuration of a parent device and a child device in a first embodiment of the obstacle notification system in FIG. 1.

FIG. 5 is a block diagram illustrating the internal configuration of a parent device and a child device in a second embodiment of the obstacle notification system in FIG. 1.

FIG. 6 is a block diagram illustrating the internal configuration of the parent device and the child device in a third embodiment of the obstacle notification system in FIG. 1.

FIG. 7 is a block diagram illustrating the internal configuration of the parent device and the child device in a fourth embodiment of the obstacle notification system in FIG. 1.

FIG. 8 is a configuration diagram showing a modification example of the obstacle notification system in FIG. 1.

FIG. 9 is a diagram illustrating a depth image obtained by an obstacle ranging sensor.

FIG. 10 is a block diagram illustrating a configuration example of hardware of a computer that executes a series of processing according to a program.

### DESCRIPTION OF EMBODIMENTS

Hereinafter, embodiments of the present technology will be described with reference to the drawings.

### Embodiment of Obstacle Notification System

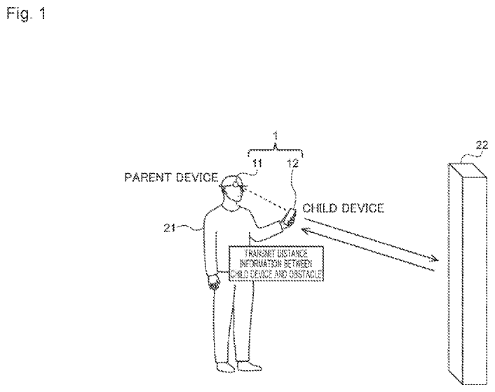

FIG. 1 is a configuration diagram showing a configuration example of an embodiment of an obstacle notification system to which the present technology is applied.

An obstacle notification system 1 of the present embodiment in FIG. 1 presents a user 21 with a notification sound corresponding to a distance or a direction of an obstacle 22 with respect to the head (ear) of the user 21 when there is the obstacle 22 that hinders walking for the user 21 such as a visually impaired person who uses this system. In the present embodiment, an object that hinders walking is referred to as an obstacle, but the present technology may be applied as a system that notifies the user 21 of the presence of any object other than such an obstacle.

The obstacle notification system 1 has a parent device 11 and a child device 12. The parent device 11 and the child device 12 are individual devices (individual objects) that are arranged at separate positions, and are communicably connected by wire or wirelessly. In the case of wireless communication, the communication may comply with arbitrary wireless communication standards such as short-range wireless communication standards such as Bluetooth (registered trademark) and ZigBee (registered trademark), wireless LAN standards such as IEEE802.11, and infrared communication standards such as IrDA.

The parent device 11 includes an audio output device such as earphones, headphones, or speakers that convert sound signals, which are electric signals, into sound waves. The audio output device may be connected to the main body of the parent device 11 by wire or wirelessly, or the main body of the parent device 11 may be incorporated into the audio output device. In the present embodiment, stereo earphones are connected to the main body of the parent device 11 by wire, and the parent device 11 is configured by the main body and the earphones of the parent device 11.

The parent device 11 is directly, or indirectly via a hat or the like, attached to the head (forehead or the like) of the user 21 so that a specific direction of the parent device 11 faces the front of the user 21. The earphones of the parent device 11 are worn on the ears of the user 21. The mounting position of the parent device 11 is not limited, and the positional relationship between the parent device 11 and the ears of the user 21 and the relationship between the specific direction of the parent device 11 and the specific direction of the head of the user 21 may be appropriately referred to in the processing of the parent device 11 or the child device 12 or the like. That is, the mounting position of the parent device 11 is not particularly limited as long as the three-dimensional position of the ears of the user 21 and the front direction of the user's head (face) (or the direction of the ears) in the coordinate system (the parent device coordinate system) fixed (set) to the parent device 11 can be specified. For example, the main body of the parent device 11 may be provided at the attachment portion of the earphone worn on the right ear or the left ear, and at the same time when the earphone is worn on the ear of the user 21, the main body of the parent device 11 may be worn on the head of the user 21 at a predetermined position and direction. Based on the three-dimensional position of the ear and the direction of the head in the parent device coordinate system at that time, processing may be performed in the parent device 11 or the child device 12.

The parent device 11 measures the distance, direction, and attitude of the child device 12 with respect to the parent device 11.

Here, for example, a three-dimensional orthogonal coordinate system fixed to the parent device 11 is referred to as a parent device coordinate system. For example, the origin of the parent device coordinate system is the coordinates indicating the three-dimensional position of the parent device 11 (the position of the head of the user 21) in the parent device coordinate system. For example, a three-dimensional orthogonal coordinate system fixed to the child device 12 will be referred to as a child device coordinate system. For example, the origin of the child device coordinate system is the coordinates indicating the three-dimensional position of the child device 12 (the position other than the head of the user 21) in the child device coordinate system.

Measurement of the distance and direction of the child device 12 with respect to the parent device 11 corresponds to measurement of the three-dimensional position (xyz coordinates) of the child device 12 in the parent device coordinate system, and corresponds to measurement of the three-dimensional position (xyz coordinates) of the origin of the child device coordinate system in the parent device coordinate system.

The attitude of the child device 12 with respect to the parent device 11 is represented by a coordinate rotation axis and a rotational movement amount around the coordinate rotation axis when the child device coordinate system is rotationally moved around a predetermined rotation axis (coordinate rotation axis) from a state where each axis of the parent device coordinate system is parallel to each axis of the child device coordinate system so as to match a current state. Measurement of the attitude of the child device 12 with respect to the parent device 11 corresponds to specifying the coordinate rotation axes and the rotational movement amounts.

Instead of the parent device 11 measuring the distance and direction of the child device 12 with respect to the parent device 11, the child device 12 may measure the distance and direction of the child device 12 with respect to the parent device 11, and the parent device 11 or the child device 12 may measure the distance and direction of the parent device 11 with respect to the child device 12. Similarly, instead of the parent device 11 measuring the attitude of the child device 12 with respect to the parent device 11, the child device 12 may measure the attitude of the child device 12 with respect to the parent device 11, and the parent device 11 or the child device 12 may measure the attitude of the parent device 11 with respect to the child device 12. That is, the relative positional relationship and attitude relationship between the parent device 11 and the child device 12 may be measured by either the parent device 11 or the child device 12, respectively.

Based on the distance of the obstacle 22 with respect to the child device 12 measured by the child device 12 and the distance, direction, and attitude of the child device 12 with respect to the parent device 11, the parent device 11 detects the distance and direction of the obstacle 22 with respect to the parent device 11 (head) and presents the user 21 with a notification sound corresponding to at least one of the detected distance and direction of the obstacle 22 through earphones. The child device 12 is not arranged at a fixed position, and is arranged at a position other than the head of the user 21 (a different position from the parent device 11), for example. For example, the user 21 may hold the child device 12 or wear the child device 12 on the hand or foot. The child device 12 may be attached to the proximal end portion or the tip portion of a white cane used by the user 21 who is visually impaired.

The child device 12 measures the distance to the child device 12, of an obstacle 22 present in a specific direction (measurement direction) with respect to the child device 12.

Here, a point where the straight line extending in the measurement direction of the child device 12 and the surface of the obstacle 22 intersect is referred to as a measurement point. The child device 12 measures the distance of the measurement point as the distance of the obstacle 22 with respect to the child device 12. Since the measurement direction in the child device coordinate system is a predetermined direction, measurement of the distance of the measurement point with respect to the child device 12 corresponds to measurement of a three-dimensional position (xyz coordinates) of the measurement point in the child device coordinate system.

<Measurement Principle of Obstacle Notification System>

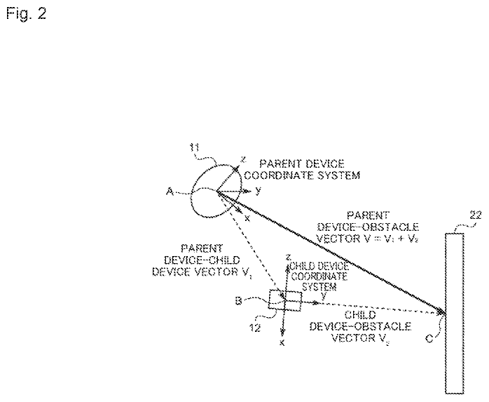

FIG. 2 is a diagram explaining the principle of measuring the three-dimensional position of the obstacle 22 (the measurement point) in the obstacle notification system 1 in FIG. 1.

In FIG. 2, a point A represents the three-dimensional position of the parent device 11. That is, the point A represents the position of the origin of the parent device coordinate system.

A point B represents the three-dimensional position of the child device 12. That is, the point B represents the position of the origin of the child device coordinate system.

A point C represents the position of the measurement point on the obstacle 22 (the point where the obstacle 22 intersects with a straight line extending in the measurement direction of the child device 12). The point C is also referred to as a measurement point C.

A parent device-child device vector v1 is a vector having the point A as a starting point and the point B as an ending point. A child device-obstacle vector v2 is a vector having the point B as the starting point and the measurement point C as the ending point. A parent device-obstacle vector V is a vector having the point as the starting point and the measurement point C as the ending point, and represents the sum of the parent device-child device vector v1 and the child device-obstacle vector v2.

The parent device 11 (or the child device 12) measures (obtains) the xyz coordinates of the point B in the parent device coordinate system by measuring the distance and direction of the child device 12 with respect to the parent device 11. The xyz coordinates of the point B in the parent device coordinate system may be calculated from the results of measuring the xyz coordinates of the point A in the child device coordinate system. It is assumed that (Bx, By, Bz) are obtained as the xyz coordinates of the point B in the parent device coordinate system as the results of the measurement. At this time, the xyz coordinate components of the parent device-child device vector v1 in the parent device coordinate system are (Bx, By, Bz).

The parent device 11 (or the child device 12) measures the attitude of the child device coordinate system in the parent device coordinate system as the attitude of the child device 12 with respect to the parent device 11. The attitude of the child device coordinate system in the parent device coordinate system can be represented by a coordinate rotation axis and the rotational movement amount (rotation angle) around the coordinate rotation axis when the child device coordinate system is rotationally moved in the direction of each axis of the child device coordinate system at the current time from a state where each axis of the xyz axes of the child device coordinate system is parallel to each axis of the xyz axes of the parent device coordinate system. The parent device 11 (or the child device 12) measures the coordinate rotation axis and the rotational movement amount around the coordinate rotation axis as the attitude of the child device coordinate system in the parent device coordinate system. However, the attitude of the child device coordinate system in the parent device coordinate system can also be expressed by other methods (Euler angles or the like). The measurement of the attitude of the child device coordinate system in the parent device coordinate system is not limited to direct measurement of the coordinate rotation axis and the rotational movement amount. Instead of measuring the attitude of the child device coordinate system in the parent device coordinate system, the attitude of the parent device coordinate system in the child device coordinate system may be measured.

The child device 12 measures the distance of the measurement point C of the obstacle 22 with respect to the child device 12, and measures (obtains) the xyz coordinates of the measurement point C in the child device coordinate system from the measured distance of the measurement point C and the measurement direction in the child device coordinate system. As a result, it is assumed that (Cx, Cy, Cz) are obtained as the xyz coordinates of the point C in the child device coordinate system. At this time, the xyz coordinate components of the child device-obstacle vector v2 in the child device coordinate system are (Cx, Cy, Cz).

The parent device 11 (or the child device 12) performs coordinate transformation from the xyz coordinate components of the child device-obstacle vector v2 in the child device coordinate system to the xyz coordinate components of the child device-obstacle vector v2 in the parent device coordinate system based on the xyz coordinate components (Cx, Cy, Cz) (the xyz coordinates of the measurement point C) of the child device-obstacle vector v2 and the attitude of the child device 12 with respect to the parent device 11. As a result, it is assumed that (Cx′, Cy′, Cz′) are obtained as the xyz coordinate components of the child device-obstacle vector v2 in the parent device coordinate system.

The parent device 11 (or the child device 12) adds the xyz coordinate components (Bx, By, Bz) of the parent device-child device vector v1 in the P(X,Y) and the xyz coordinate components (Cx′, Cy′, Cz′) of the child device-obstacle vector v2 in the parent device coordinate system for each of the xyz coordinate components to calculate the xyz coordinate components of the parent device-obstacle vector V (=v1+v2) in the parent device coordinate system. As a result, (Bx+Cx′, By+Cy′, Bz+Cz′) are obtained as the xyz coordinate components of the parent device-obstacle vector V in the parent device coordinate system. In this way, it is assumed that the distance and direction of the obstacle 22 (the measurement point C) with respect to the parent device 11 (point A) are obtained as the parent device-obstacle vector V.

The parent device 11 generates a notification sound corresponding to the obtained parent device-obstacle vector V and presents the same to the user 21. As a simple example of the notification sound, for example, the smaller the magnitude of the parent device-obstacle vector V, that is, the smaller the distance between the head of the user 21 and the measurement point C of the obstacle 22, the smaller the volume of the notification sound.

According to the obstacle notification system 1 in FIG. 1, for example, even when the child device 12 having a range-finding function of measuring the distance to an obstacle (the measurement point C) is arranged in an arbitrary portion other than the head such as the hand of the user 21 or the tip of a white cane, a notification sound corresponding to the distance and direction of the obstacle (the measurement point C) with respect to the head is presented to the user 21. Therefore, the distance and direction based on the head of the user 21 can be measured even for an obstacle (the measurement point C) present at a distance where the same cannot be measured from the head of the user 21. Regardless of the position of the child device 12 having the range-finding function with respect to the head of the user 21, since a notification sound corresponding to the distance and direction of the obstacle (the measurement point C) based on the head of the user 21 is always presented to the user 21, the user 21 can reliably and stably perceive obstacles present in the surroundings.

<Processing Procedure of Obstacle Notification System>

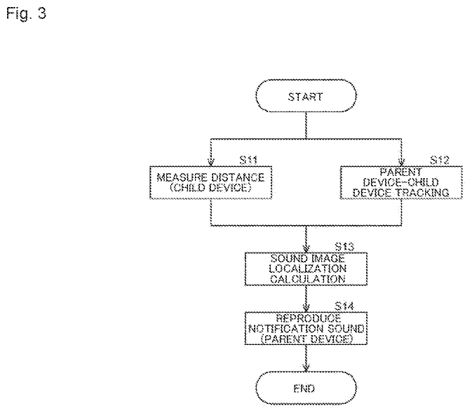

FIG. 3 is a flowchart illustrating the processing procedure of the obstacle notification system 1 in FIG. 1.

In step S11, the obstacle notification system 1 (the child device 12) measures the distance to the obstacle 22 (the measurement point C) present in the measurement direction. The processing proceeds from step S11 to step S13.

In step S12, the obstacle notification system 1 (the parent device 11 or the child device 12) measures the relative three-dimensional position and attitude between the parent device 11 and child device 12. The measurement of the relative distance, direction, and attitude between the parent device 11 and the child device 12 is referred to as tracking. Step S12 is performed in parallel with step S11. The processing proceeds from step S12 to step S13.

In step S13, the obstacle notification system 1 (the parent device 11 or the child device 12) calculates sound image localization based on the distance to the obstacle 22 (the measurement point C), which is the measurement result of step S11, and the relative distance, direction, and attitude between the parent device 11 and the child device 12 which is the measurement result of step S12.

Calculation of sound image localization means generating a notification sound for allowing the user to perceive the position of a sound image. The obstacle notification system 1 calculates the distance and direction of the obstacle 22 (the measurement point C) with respect to the parent device 11 described in FIG. 2 based on the distance to the obstacle 22 (the measurement point C) which is the measurement result of step S11 and the relative distance, direction, and attitude between the parent device 11 and the child device 12, which is the measurement result of step S12. The obstacle notification system 1 generates right and left notification sounds for allowing the user to perceive the three-dimensional position of the obstacle 22 (the measurement point C) specified by the calculated distance and direction as the position of the sound image. The processing proceeds from step S13 to step S14.

In step S14, the obstacle notification system 1 (the parent device 11) outputs the notification sound generated in step S13 from earphones and presents the same to the user 21.

Through the above-described processing, the obstacle notification system 1 generates a notification sound that allows the user to perceive the distance and direction of the obstacle (the measurement point C) with respect to the head as sound image localization, and presents the same to the user 21.

### First Embodiment of Obstacle Notification System 1

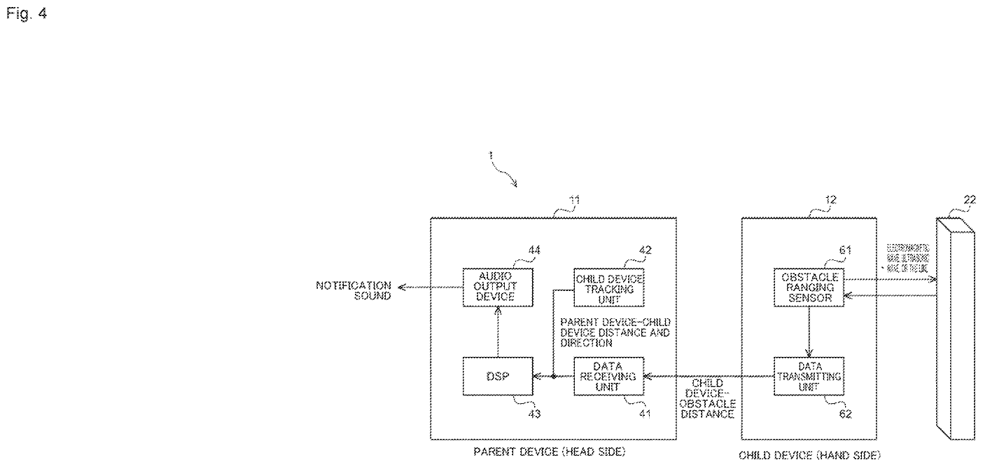

FIG. 4 is a block diagram illustrating the internal configuration of the parent device 11 and the child device 12 in the first embodiment of the obstacle notification system 1 in FIG. 1.

The parent device 11 has a data receiving unit 41, a child device tracking unit 42, a digital signal processor (DSP) 43, and an audio output unit 44. The child device 12 has an obstacle ranging sensor 61 and a data transmitting unit 62.

In the parent device 11, the data receiving unit 41 performs wired or wireless communication with the data transmitting unit 62 of the child device 12. The data receiving unit 41 and the data transmitting unit 62 may be data transceiving units that transmit and receive data in both directions. The data receiving unit 41 obtains the child device-obstacle distance measured by the obstacle ranging sensor 61 of the child device 12 from the data transmitting unit 62. The child device-obstacle distance is the distance from the child device 12 to the measurement point C of the obstacle 22. The data receiving unit 41 supplies the obtained child device-obstacle distance to the DSP 43. The child device-obstacle distance corresponds to the magnitude of the child device-obstacle vector v2 described in FIG. 2. The child device tracking unit 42 tracks the child device 12, and measures the distance and direction of the child device 12 with respect to the parent device 11 (parent device-child device distance and direction) and the attitude of the child device 12 with respect to the parent device 11 (the parent device-child device attitude). The parent device-child device distance and direction represent the magnitude and direction of the parent device-child device vector v1 in the parent device coordinate system described in FIG. 2, and corresponds to the parent device-child device vector v1. The measurement of the parent device-child device distance and direction corresponds to the measurement of the xyz coordinate components (Bx, By, Bz) of the program distribution server v1 of the child device 12 in the parent device coordinate system described in FIG. 2. The child device tracking unit 42 is not limited to measuring the magnitude and direction values of the parent device-child device vector v1 themselves.

The measurement of the parent device-child device attitude corresponds to the measurement of the rotational movement amount of the child device 12 in the parent device coordinate system from the reference state, and corresponds to the measurement of the coordinate rotation axis and the rotational movement amount in the rotational movement of the child device coordinate system with respect to the parent device coordinate system described in FIG. 2.

The child device tracking unit 42 supplies the parent device-child device distance and direction and the parent device-child device attitude obtained by the measurement to the DSP 43. The details of the measurement of the parent device-child device distance and direction and the parent device-child device attitude will be described later.

The DSP 43 calculates sound image localization based on the child device-obstacle distance from the data receiving unit 41 and the parent device-child device distance and direction and the parent device-child device attitude from the child device tracking unit 42. The DSP 43 generates a notification sound (notification sound signal) to be presented to the user 21 by sound image localization calculation. Processing for sound image localization calculation will be described later. The DSP 43 supplies the notification sound generated by the sound image localization calculation to the audio output unit 44.

When the audio output unit 44 of the parent device 11 is a stereo-compatible earphone, headphone, or speaker, the DSP 43 outputs a stereo (2ch) notification sound composed of a right (right-ear) notification sound and a left (left-ear) notification sound. If the audio output unit 44 is a monaural-compatible earphone, headphone, or speaker, the DSP 43 generates a monaural (1ch) notification sound.

However, the DSP 43 may generate a monaural notification sound even if the audio output unit 44 supports stereo, or may generate a stereo notification sound even if the audio output unit 44 supports monaural. That is, the number of channels of the audio output unit 44 and the number of channels of the notification sound generated by the DSP 43 may not be necessarily consistent. The inconsistency in the number of channels between the DSP 43 and the audio output unit 44 can be adjusted by integrating notification sounds of multiple channels, using a notification sound of one channel in multiple channels, or the like.

In the present embodiment, it is assumed that the audio output unit 44 is a stereo earphone, and the DSP 43 generates a stereo notification sound composed of a right notification sound and a left notification sound.

The audio output unit 44 converts the notification sound (notification sound signal) from the DSP 43 from an electric signal into a sound wave by the earphones worn on both ears of the user 21, and outputs the sound wave.

In the child device 12, the obstacle ranging sensor 61 emits a measurement wave such as an ultrasonic wave or an electromagnetic wave in a specific direction (measurement direction) to the child device 12, and detects a measurement wave reflected from the obstacle 22 which is an obstacle present in the measurement direction. The obstacle ranging sensor 61 measures the distance to the position (the measurement point C in FIG. 2) where the measurement wave is reflected by the obstacle 22 according to the ToF (Time of Flight) principle. The obstacle ranging sensor 61 may be any known ranging sensor. The obstacle ranging sensor 61 supplies the data transmitting unit 62 with the child device-obstacle distance, which is the distance to the measuring point C of the obstacle 22 obtained by the measurement. The child device-obstacle distance corresponds to the magnitude of the child device-obstacle vector v2 described in FIG. 2.

The data transmitting unit 62 performs wired or wireless communication with the data receiving unit 41 of the parent device 11. The data transmitting unit 62 transmits the child device-obstacle distance from the obstacle ranging sensor 61 to the data receiving unit 41.

(Sound Image Localization Calculation by DSP)

The sound image localization calculation processing of the DSP 43 will be described. The DSP 43 of the parent device 11 executes obstacle position calculation processing for calculating the distance and direction (three-dimensional position in the parent device coordinate system) of the measurement point C of the obstacle 22 with respect to the head of the user 21 as sound image localization calculation processing.

After the obstacle position calculation processing, the DSP 43 executes notification sound generation processing as the sound image localization calculation processing. In the notification sound generation processing, the DSP 43 generates a right (right ear) notification sound and a left (left ear) notification sound that propagate to the right and left ears of the user 21 when a sound is virtually emitted using the three-dimensional position of the measurement point C specified by the distance and direction of the measurement point C of the obstacle 22 calculated in the obstacle position calculation processing as the position of the sound source.

First, the obstacle position calculation processing in the sound image localization calculation of the DSP 43 will be described.

In the obstacle position calculation processing, the DSP 43 calculates the parent device-obstacle vector V described in FIG. 2 based on the child device-obstacle distance from the data receiving unit 41 and the parent device-child device distance and direction and the parent device-child device attitude from the child device tracking unit 42. In this way, the DSP 43 calculates the distance and direction of the measurement point C of the obstacle 22 with respect to the three-dimensional position of the parent device 11, and specifies the three-dimensional position of the measurement point C.

That is, the xyz coordinate components (Bx, By, Bz) of the parent device-child device vector v1 in the parent device coordinate system described in FIG. 2 are obtained from the parent device-child device distance and direction from the child device tracking unit 42.

The xyz coordinate components (Cx, Cy, Cz) of the child device-obstacle vector v2 in the child device coordinate system described in FIG. 2 are obtained from the child device-obstacle distance from the data receiving unit 41. It is assumed that the DSP 43 has previously grasped the measurement direction in the child device coordinate system.

The xyz coordinate components (Cx′, Cy′, Cz′) of the child device-obstacle vector v2 in the parent device coordinate system are obtained from the parent device-child device attitude, which is the attitude of the child device 12 with respect to the parent device 11, and the xyz coordinate components (Cx, Cy, Cz) of the child device-obstacle vector v2 in the child device coordinate system.

As a result, the xyz coordinate components (Bx+Cx′, By+Cy′, Bz+Cz′) of the parent device-obstacle vector V in the parent device coordinate system are obtained from the xyz coordinate components (Bx, By, Bz) of the parent device-child device vector v1 in the parent device coordinate system and the xyz coordinate components (Cx′, Cy′, Cz′) of the child device-obstacle vector v2 in the parent device coordinate system. (Bx+Cx′, By+Cy′, Bz+Cz′) are the xyz coordinates representing the three-dimensional position of the measurement point C of the obstacle 22 in the parent device coordinate system, and the three-dimensional position of the measurement point C is specified.

Next, the notification sound generation processing in the sound image localization calculation by the DSP 43 will be described.

In the notification sound generation processing, the DSP 43 performs notification sound generation processing on an original sound (original sound signal indicating the original sound) that is the source of the notification sound, and generates a notification sound (notification sound signal indicating the notification sound) to be presented to the user 21.

In the notification sound generation processing, the DSP 43 uses the three-dimensional position (Bx+Cx′, By+Cy′, Bz+Cz′) of the measurement point C of the obstacle 22 calculated by the obstacle position calculation processing as the position of a virtual sound source. The DSP 43 assumes that the original sound, which is the source of an obstacle notification sound, is emitted from the position of a virtual sound source.

A sound signal (original sound signal) serving as the original sound may be, for example, a sound signal stored in advance in a memory (not shown) that can be referred to by the DPS 43. The original sound signal stored in the memory may be a sound signal such as a continuous or intermittent alarm sound specialized as a notification sound, or a sound signal such as music not specialized as a notification sound. The original sound signal may be a sound signal such as music supplied as streaming from an external device connected to the parent device 11 or the child device 12 via a network such as the Internet. The original sound signal may be a sound signal of environmental sound collected by a microphone (not shown). When the sound signal in the memory or the sound signal supplied as streaming is used as the original sound signal, if the earphone worn by the user 21 is an open-type earphone, the user 21 can hear the environmental sound and the notification sound at the same time.

The DSP 43 generates the sound when the original sound emitted from the measurement point C which is the position of a sound source propagates through the air and reaches the right and left ears of the user 21 as the right (right-ear) notification sound and the left (left-ear) notification sound. Only the generation of the right notification sound will be described below, and the description of the left notification sound will be omitted assuming that it is generated in the same manner as the right notification sound.

When generating the notification sound from the original sound, the DSP 43 sets the position and direction of the right ear (and the left ear) in the parent device coordinate system. For example, it is assumed that the user 21 wears the parent device 11 on the head (near the forehead) with the specific direction (reference direction) of the parent device 11 facing the front of the head (face) as a rule of use. At this time, the DSP 43 sets the three-dimensional position (point A) of the parent device 11, which is the starting point of the parent device-obstacle vector V in the parent device coordinate system described in FIG. 2, that is, the origin of the parent device coordinate system, as the position of the head (forehead) of the user 21. The DSP 43 sets the specific direction (reference direction) of the parent device 11 expressed in the parent device coordinate system as the front direction of the head of the user 21, and determines the three-dimensional position and direction (up-down, left-right, front-back direction) of the right ear (and left ear) of the user 21 in the parent device coordinate system from the origin and the reference direction in the parent device coordinate system based on the average human head structure. When the three-dimensional position of the right ear in the parent device coordinate system is set, the DSP 43 sets a line segment connecting the three-dimensional position of the measurement point C, which is the position of the sound source, and the three-dimensional position of the right ear in the parent device coordinate system as the propagation path of the original sound toward the right ear. However, the three-dimensional positions of the right ear and the left ear may be the same position in front of the head (the origin of the parent device coordinate system). The three-dimensional position of the sound source that emits the original sound may be a position different from the measurement point C. For example, the position of the sound source that emits the original sound may be the three-dimensional position (the origin of the parent device coordinate system) of the parent device 11 (point A). In this case, it is assumed that the original sound emitted from the sound source is reflected at the measurement point C using the measurement point C as a reflection position (scattering position) and propagates to the right ear. At this time, the propagation path of the original sound toward the right ear is composed of a line segment connecting the three-dimensional position of the parent device 11 (point A) and the three-dimensional position of the measurement point C, and a line segment connecting the three-dimensional position of the measurement point C and the three-dimensional position of the right ear. In this case, a notification sound is generated in which the three-dimensional position of the measurement point C where the original sound is reflected is perceived as the position of the sound image.

Considering that the mounting position of the parent device 11 on the user 21 is not limited to the head (near the forehead), the point A, which is the origin of the parent device coordinate system in FIG. 2, may be interpreted to represent the three-dimensional position of the head of the user 21 rather than the three-dimensional position of the parent device 11. In this case, when the parent device-child device distance and direction, that is, the magnitude and direction of the parent device-child device vector v1 in the parent device coordinate system described in FIG. 2 are obtained by the measurement of the child device tracking unit 42, the child device tracking unit 42 corrects the measurement result based on the positional relationship between the mounting position of the parent device 11 and the head of the user 21, obtains a vector directed from the three-dimensional position of the head toward the three-dimensional position of the child device 12. The DSP 43 regards the vector as the parent device-child device vector v1 and performs the sound image localization calculation processing.

The DSP 43 adds changes (modulation) to the right notification sound by at least one of a head-related transfer function, a delay action, and a volume attenuation action according to the propagation path of the original sound toward the right ear so that a difference from the left notification sound is created. As a result, a notification sound is generated that allows the user to perceive a sound image at the three-dimensional position of the measurement point C of the obstacle 22. In the following, a case where changes are added to the original sound by all the elements will be described.

The head-related transfer function indicates the transfer characteristics around the head for each arrival direction of sound that arrives at the ear along the propagation path. The DSP 43 has a head-related transfer function for each arrival direction generated in advance assuming an average human body structure around the ear. The same head-related transfer function may be associated with an approximate arrival direction, and the arrival direction not associated with the head-related transfer function may be estimated from the head-related transfer function of the approximate arrival direction by interpolation processing or the like. The DSP 43 convolves the original sound (original sound signal) with a head-related transfer function corresponding to the propagation path of the original sound emitted from the three-dimensional position of the measurement point C of the obstacle 22, which is the position of the sound image, to the right ear (convolutional integration). The head-related transfer function corresponding to the propagation path to the right ear indicates the head-related transfer function corresponding to the arrival direction of the sound arriving at the right ear along the propagation path. As a result, a notification sound is generated when the original sound emitted from the measurement point C is changed due to the influence of the body shape around the head before reaching the right ear.

The DSP 43 further adds changes to the notification sound generated by convolving the head-related transfer function and the original sound signal by a delay action.

The delay action is a time delay action of the notification sound with respect to the original sound caused by the propagation time corresponding to the length of the propagation path of the original sound emitted from the measurement point C until it reaches the right ear. By the processing of the delay action, the DSP 43 increases the delay (phase delay) of the notification sound signal after change with respect to the notification sound signal before change due to the delay action as the length of the propagation path increases.

The DSP 43 further adds changes to the notification sound generated by the convolution of the head-related transfer function and the original sound signal and the delay action by a volume attenuation action. The volume attenuation action is the action of amplitude (volume) attenuation occurring in the propagation path of the original sound signal emitted from the measurement point C until it reaches the right ear. By the processing of the volume attenuation action, the DSP 43 increases the attenuation of the amplitude of the notification sound signal (decreases the amplitude) as the length of the propagation path increases. The DSP 43 supplies the audio output unit 44 with the right notification sound obtained by adding changes (modulation) to the original sound by these head-related transfer function, delay action, and volume attenuation action, and the left notification sound obtained by adding changes (modulation) in the same way as the right notification sound. However, the order of adding changes to the original sound by the head-related transfer function, delay action, and volume attenuation action is not limited to a specific order.

When an original sound signal of multiple channels of 2ch or more is used as the original sound, the DSP 43 may generate a notification sound signal for each channel from the original sound signal of each channel, or may integrate the original sound signal of each channel into one original sound signal and generate notification sound signals for a predetermined number of channels from the integrated original sound signal. For example, when a stereo original sound signal composed of the right and left original sound signals is used as the original sound, in one aspect, the DSP 43 generates a right notification sound signal from the right original sound signal, and generates a left notification sound signal from the left original sound signal. In another aspect, the DSP 43 integrates the right original sound signal and the left original sound signal to generate a monaural original sound signal, and generates the right notification sound signal and the left notification sound signal from the monaural original sound signal.

If the obstacle is not present within a predetermined distance from the head, the DSP 43 supplies a notification sound for allowing the user 21 to perceive that there is no object as the notification sound to be supplied to the audio output unit 44. As a notification sound for allowing the user to perceive that there is no obstacle, the DSP 43 supplies the audio output unit 44 with, for example, silence, an original sound without sound image localization (original sound itself), or a notification sound obtained by adding changes to the original sound so that the sound image faces the front.

When the DSP 43 supplies the audio output unit 44 with an original sound such as music that is different from the original sound that is the source of the notification sound, regardless of the notification sound, and supplies the notification sound to the audio output unit 44, the DSP 43 may synthesize the original sound and the notification sound supplied to the audio output unit 44 and supply the synthesized sound to the audio output unit 44.

According to the sound image localization calculation processing of the DSP 43, for example, even when the child device 12 having the range-finding function for measuring the distance to the obstacle (the measurement point C) is arranged in an arbitrary part other than the head such as the hand of the user 21 or the tip of a white cane, the user 21 is presented with a notification sound that allows the user 21 to perceive the position corresponding to the distance and direction of the obstacle (the measurement point C) with respect to the head as the position of the sound image. Therefore, the distance and direction from the head of the user 21 can be measured even for an obstacle (measuring point C) present at a distance where the same cannot be measured from the head of the user 21. Regardless of the position of the child device 12 having a range-finding function with respect to the head of the user 21, since the notification sound for allowing the user to perceive the distance and direction of the obstacle (measurement point C) based on the head of the user 21 is always presented to the user 21, the user 21 can reliably and stably perceive obstacles present in the surroundings.

The DSP 43 generates a notification sound corresponding to the distance and direction of the obstacle (the measurement point C) with respect to the head (point A) of the user 21, but the present invention is not limited to this. The DSP 43 may use a position different from the position of the child device 12, such as an arbitrary part of the body of the user 21, as a reference position, instead of using the head as a reference, and generate a communication sound corresponding to the distance and direction of the obstacle (the measurement point C) with respect to the reference position.

The present technology is not limited to the case where the DSP 43 generates a notification sound having sound image localization. The present technology includes the case where the DSP 43 generates a notification sound based on at least one of the distance and direction of the obstacle (the measurement point C) with respect to the head (reference position). For example, the shorter the distance of the obstacle (the measurement point C) with respect to the head (reference position), the shorter the pulse period of a pulse-shaped notification sound (notification sound signal) that is generated intermittently and periodically. Alternatively, the tone color of the notification sound may be changed according to the distance of the obstacle (the measurement point C) with respect to the head (reference position). For example, when the pulse period of the pulse-shaped notification sound (notification sound signal) may be shortened as the direction of the obstacle (the measurement point C) with respect to the head (reference position) is closer to the rear side than the front side of the head. In the present technology, instead of the notification sound (notification sound signal), a signal (notification signal) based on vibration or light corresponding to at least one of the distance and direction of the obstacle (the measurement point C) with respect to the head (reference position) may be presented to the user 21.

(Parent Device-Child Device Tracking) Next, tracking between the parent device 11 and the child device 12 (parent device-child device tracking) will be described.

In the obstacle position calculation processing in the DPS 43, the parent device-obstacle vector V representing the distance and direction of the measurement point C of the obstacle 22 with respect to the three-dimensional position of the parent device 11 is calculated as the xyz coordinate components (Bx+Cx′, By+Cy′, Bz+Cz′) in the parent device coordinate system.

Therefore, in the obstacle position calculation processing, the DSP 43 obtains the parent device-child device distance and direction (the parent device-child device vector v1) and the parent device-child device attitude from the child device tracking unit 42. The parent device-child device distance and direction are used to calculate the xyz coordinate components (Bx, By, Bz) of the parent device-child device vector v1 in the parent device coordinate system. The parent device-child device attitude is used to calculate the xyz coordinate components (Cx′, Cy′, Cz′) of the child device-obstacle vector v2 in the parent device coordinate system from the xyz coordinate components (Cx, Cy, Cz) of the child device-obstacle vector v2 in the child device coordinate system by coordinate transformation. (Cx, Cy, Cz) is obtained from the child device-obstacle distance from the obstacle ranging sensor 61 and the predetermined measurement direction in the child device coordinate system. In such an obstacle position calculation processing, the parent device-obstacle vector V calculated finally is not limited to the case where it is obtained as the xyz coordinate components in the parent device coordinate system. The parent device-obstacle vector V may be obtained as the coordinate components in another coordinate system (such as a polar coordinate system) fixed to the parent device 11 or may be obtained as the coordinate components in an arbitrary coordinate system fixed to the child device 12, the ground, the obstacle 22, or the like. Therefore, the parent device-child device vector v1 and the parent device-child device attitude obtained to calculate the parent device-obstacle vector V may be information indicating the relative positional relationship between the parent device 11 and the child device 12 and the relative attitude between the parent device 11 and the child device 12 (the attitude between the coordinate system fixed to the parent device and the coordinate system fixed to the child device). These pieces of information are not limited to being obtained by the child device tracking unit 42 mounted on the parent device 11.

In the obstacle notification system 1 of the present embodiment, any tracking device of known tracking systems can be employed as the tracking device for measuring the relative positional relationship and direction between the parent device 11 and the child device 12. Examples of the tracking device of the known tracking system include magnetic, optical, wireless (radio wave), and inertial tracking systems. A case where a magnetic or optical tracking device is employed in the obstacle notification system 1 will be briefly described.

A magnetic tracking device has a transmitter that generates a magnetic field and a receiver that detects changes in the magnetic field. The transmitter and the receiver each have a 3-way quadrature coil. The transmitter excites each quadrature coil in turn and the receiver measures the electromotive force generated in each quadrature coil to detect the distance, direction, and attitude of the receiver with respect to the transmitter. Regarding magnetic tracking, reference can be made to the literature “Ke-Yu Chen et al., “Finexus: Tracking Precise Motions of Multiple Fingertips Using Magnetic Sensing”, Proceedings of the 2016 CHI Conference on Human Factors in Computing Systems, (US), May 2016, p. 1504-1514″, for example.

As an example of the case where the obstacle notification system 1 employs a magnetic tracking device, the transmitter is mounted on the parent device 11 and the receiver is mounted on the child device 12. In this case, the tracking processing unit that calculates the distance, direction, and attitude of the child device 12 with respect to the parent device 11 based on the information obtained by the receiver may be arranged in either the parent device 11 or the child device 12. When the tracking processing unit is arranged in the parent device 11, the tracking processing unit obtains the information obtained by the receiver of the child device 12 from the child device 12. It is assumed that data transmission between the parent device 11 and the child device 12 is performed in both directions between the data transceiving unit of the parent device 11 including the data receiving unit 41 in FIG. 4 and the data transceiving unit including the data transmitting unit 62 in FIG. 4 (the same applies below). When the tracking processing unit is arranged in the child device 12, the tracking processing unit obtains the information obtained by the receiver of the child device 12 by information transmission within the child device 12.

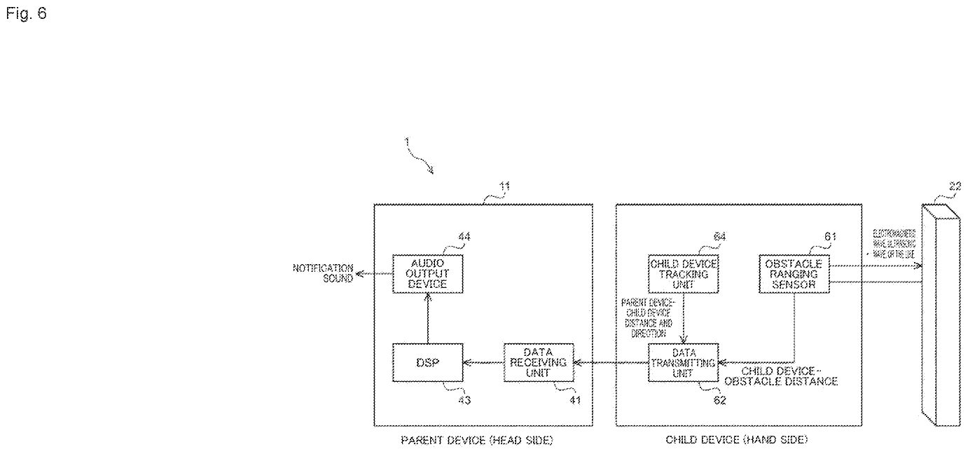

The child device tracking unit 42 arranged in the parent device 11 in FIG. 4 corresponds to the transmitter and the tracking processing unit when the tracking processing unit is arranged in the parent device 11. The child device tracking unit 64 arranged in the child device 12 in FIG. 6 described later corresponds to the receiver and the tracking processing unit when the tracking processing unit is arranged in the child device 12.

As another example of the case where the obstacle notification system 1 employs a magnetic tracking device, the transmitter is mounted on the child device 12 and the receiver is mounted on the parent device 11. In this case, the tracking processing unit that calculates the distance, direction, and attitude of the parent device 11 with respect to the child device 12 based on the information obtained by the receiver may be arranged in either the parent device 11 or the child device 12. When the tracking processing unit is arranged in the child device 12, the tracking processing unit obtains the information obtained by the receiver of the parent device 11 from the parent device 11. When the tracking processing unit is arranged in the parent device 11, the tracking processing unit obtains the information obtained by the receiver of the parent device 11 by information transmission within the parent device 11.

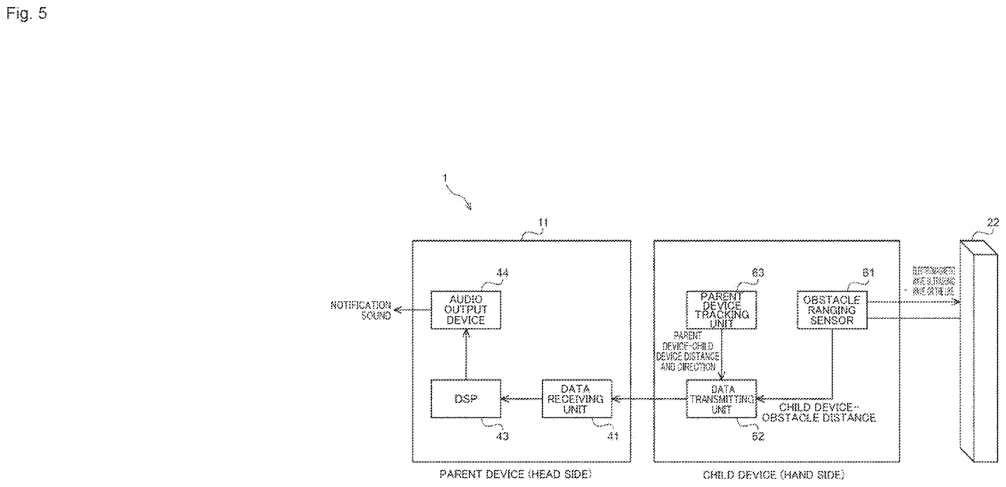

The parent device tracking unit 63 arranged in the child device 12 in FIG. 5 described later corresponds to the transmitter and the tracking processing unit when the child device 12 is arranged in the tracking processing unit.

Optical tracking devices include a marker type and an image type. A marker-based tracking device has a plurality of reflective markers attached to a tracking target, and a plurality of infrared cameras installed in a tracking-side device that tracks the tracking target. The infrared camera has, for example, an infrared irradiation function. Each infrared camera emits infrared rays and photographs the reflective markers. The tracking processing unit of the tracking device uses the principle of triangulation to detect the distance and direction of each reflective marker with respect to the tracking-side device by detecting the position of the reflective marker in the image captured by each infrared camera. In this way, the distance, direction, and attitude of the tracking target with respect to the tracking-side device are detected. Regarding the marker-based tracking, reference can be made to the literature “Shangchen Han et al., “Online Optical Marker-based Hand Tracking with Deep Labels”, ACM Transactions on Graphics, 2018, vol. 37, No. 4, p. 1-10″, for example.

As an example of a case where a marker-based tracking device is adopted in the obstacle notification system 1, the parent device 11 is a tracking-side device having a plurality of infrared cameras, and the child device 12 is a tracking target to which a plurality of reflective markers are attached. In this case, the tracking processing unit that calculates the distance, direction, and attitude of the child device 12 with respect to the parent device 11 based on the images captured by a plurality of infrared cameras may be arranged in either the parent device 11 or the child device 12. When the tracking processing unit is arranged in the child device 12, the tracking processing unit obtains the images captured by the plurality of infrared cameras of the parent device 11 from the parent device 11. When the tracking processing unit is arranged in parent device 11, the tracking processing unit obtains the images captured by the plurality of infrared cameras of the parent device 11 by information transmission within the parent device 11.

The child device tracking unit 42 arranged in the parent device 11 in FIG. 4 corresponds to a plurality of infrared cameras and a tracking processing unit when the tracking processing unit is arranged in the parent device 11. The child device tracking unit 64 arranged in the child device 12 in FIG. 6 described later corresponds to a reflective marker and a tracking processing unit when the tracking processing unit is arranged in the child device 12.

As another example of a case where a marker-based tracking device is adopted in the obstacle notification system 1, the parent device 11 is a tracking target to which a plurality of reflective markers is attached, and the child device 12 is a tracking-side device having a plurality of infrared cameras. In this case, the tracking processing unit that calculates the distance, direction, and attitude of the parent device 11 with respect to the child device 12 based on the images captured by the plurality of infrared cameras may be arranged in either the parent device 11 or the child device 12. When the tracking processing unit is arranged in the parent device 11, the tracking processing unit obtains the images captured by the plurality of infrared cameras of the child device 12 from the child device 12. When the tracking processing unit is arranged in the child device 12, the tracking processing unit obtains the images captured by the plurality of infrared cameras of the child device 12 by information transmission within the child device 12.

The parent device tracking unit 63 arranged in the child device 12 in FIG. 5 corresponds to a plurality of infrared cameras and a tracking processing unit when the tracking processing unit is arranged in the child device 12.

The image-based tracking device does not have a marker attached to the tracking target unlike the marker-based tracking device, and has a plurality of cameras installed in a tracking-side device that tracks the tracking target. Each camera photographs a tracking target. The tracking processing unit of the tracking device detects the positions of the feature points of the tracking target in the images captured by each camera, and uses the principle of triangulation to detect the distance and direction of each feature point of the tracking target with respect to the tracking device. In this way, the relative distance, direction, and attitude of the tracking target with respect to the tracking device are detected.

As an example of a case where an image-based tracking device is adopted in the obstacle notification system 1, the parent device 11 is a tracking-side device having a plurality of cameras, and the child device 12 is a tracking target. In this case, the tracking processing unit that calculates the distance, direction, and attitude of the child device 12 with respect to the parent device 11 based on the images captured by a plurality of cameras may be arranged in either the parent device 11 or the child device 12. When the tracking processing unit is arranged in the child device 12, the tracking processing unit obtains the images captured by the plurality of cameras of the parent device 11 from the parent device 11. When the tracking processing unit is arranged in parent device 11, the tracking processing unit obtains the images captured by the plurality of cameras of the parent device 11 by information transmission within the parent device 11.

The child device tracking unit 42 arranged in the parent device 11 in FIG. 4 corresponds to a plurality of cameras and a tracking processing unit when the tracking processing unit is arranged in the parent device 11. The child device tracking unit 64 arranged in the child device 12 in FIG. 6 described later corresponds to a tracking processing unit when the tracking processing unit is arranged in the child device 12.

As another example of a case where an image-based tracking device is adopted in the obstacle notification system 1, the parent device 11 is the tracking target, and the child device 12 is the tracking-side device having a plurality of cameras. In this case, the tracking processing unit that calculates the distance, direction, and attitude of the parent device 11 with respect to the child device 12 based on the images captured by a plurality of cameras may be arranged in either the parent device 11 or the child device 12. When the tracking processing unit is arranged in the parent device 11, the tracking processing unit obtains the images captured by the plurality of cameras of the child device 12 from the child device 12. When the tracking processing unit is arranged in the child device 12, the tracking processing unit obtains the images captured by the plurality of cameras of the child device 12 by information transmission within the child device 12.

The parent device tracking unit 63 arranged in the child device 12 in FIG. 5 corresponds to a plurality of cameras and a tracking processing unit when the tracking processing unit is arranged in the child device 12.

Supplementally, a case where the obstacle notification system 1 employs a tracking device of a type other than the magnetic type and the optical type will be described.

When a wireless tracking device is adopted in the obstacle notification system 1, one of the parent device 11 and the child device 12 is a tracking-side device equipped with a transmitter that emits radio waves, and the other is a tracking target equipped with an antenna that receives the the radio waves. Regarding wireless tracking, there are methods such as AoA (Angle of Arrival) and TDOA (Time Difference of Arrival), and reference can be made to JP 2005-326419 A, for example.

When a wireless tracking device is adopted in the obstacle notification system 1, each of the parent device 11 and the child device 12 is equipped with an acceleration sensor and an angular velocity sensor (IMU: inertial measurement unit), and a tracking processing unit mounted on either one of the parent device 11 and the child device 12 obtains an output signal from the IMU of each of the parent device 11 and the child device 12, and detects the distance, direction, and attitude between the parent device 11 and the child device 12. In this case, for example, calibration, parameterization, or the like is performed on the initial position such as initializing the IMU with the parent device 11 and the child device 12 facing directly downward, or fixing the distance when the child device 12 is separated from the parent device 11 to the maximum limit to a predetermined distance. Regarding inertial tracking, reference can be made to the literature “J. Connolly et al., “IMU Sensor-Based Electronic Goniometric Glove for Clinical Finger Movement Analysis”, IEEE Sensors Journal, 2018, vol. 18, No. 3, p. 1273-1281″, for example.

### Second Embodiment of Obstacle Notification System 1

FIG. 5 is a block diagram illustrating the internal configuration of the parent device 11 and the child device 12 in the second embodiment of the obstacle notification system 1 in FIG. 1. The same reference numerals are assigned to the parts common to those of the parent device 11 and the child device 12 in FIG. 4, and the description thereof will be omitted as appropriate.

The parent device 11 shown in FIG. 5 has a data receiving unit 41, a DSP 43, and an audio output unit 44. The child device 12 in FIG. 5 has an obstacle ranging sensor 61, a data transmitting unit 62, and a parent device tracking unit 63. Therefore, the parent device 11 in FIG. 5 is common to the parent device 11 in FIG. 4 in that it has the data receiving unit 41, the DSP 43, and the audio output unit 44. The child device 12 in FIG. 5 is common to the child device 12 in FIG. 4 in that it has the obstacle ranging sensor 61 and the data transmitting unit 62.

The parent device 11 in FIG. 5 is different from the parent device 11 in FIG. 4 in that it does not have the child device tracking unit 42. The child device 12 in FIG. 5 is different from the child device 12 in FIG. 4 in that a parent device tracking unit 63 is newly provided.

In FIG. 5, the parent device tracking unit 63 tracks the parent device 11, and measures the distance and direction (parent device-child device distance and direction) of the parent device 11 with respect to the child device 12 and the attitude (parent device-child device attitude) of the parent device 11 with respect to the child device 12. The parent device tracking unit 63 supplies the parent device-child device distance and direction and the parent device-child device attitude obtained by the measurement to the DSP 43 of the parent device 11 via the data transmitting unit 62 and the data receiving unit 41 as the information indicating the relative positional relationship and attitude between the parent device 11 and the child device 12.

Instead of obtaining the parent device-child device distance and direction from the child device tracking unit 42 in FIG. 4, the DSP 43 obtains the parent device-child device distance and direction and the parent device-child device attitude from the parent device tracking unit 63 in FIG. 5 as the information indicating the relative positional relationship and attitude between the parent device 11 and the child device 12.

As a result, in the obstacle position calculation processing, as in the case in FIG. 4, the DPS 43 calculates the distance and direction (parent device-obstacle vector V) of the measurement point C of the obstacle 22 with respect to the three-dimensional position of the parent device 11 based on the child device-obstacle distance obtained from the obstacle ranging sensor 61 and the parent device-child device distance and direction and the parent device-child device attitude from the parent device tracking unit 63.

According to the second embodiment of the obstacle notification system 1 described above, the parent device 11 can be made smaller than the first embodiment, and the burden on the head of the user 21 can be reduced. Therefore, the distance and direction from the head of the user 21 can be measured even for an obstacle (the measurement point C) present at a distance where the same cannot be measured from the head of the user 21. Regardless of the position of the child device 12 having the range-finding function with respect to the head of the user 21, since a notification sound for allowing the user 21 to perceive the distance and direction of the obstacle (the measurement point C) based on the head of the user 21 at the position of the sound image is always presented to the user 21, the user 21 can reliably and stably perceive obstacles present in the surroundings.

### Third Embodiment of Obstacle Notification System 1

FIG. 6 is a block diagram illustrating the internal configuration of the parent device 11 and the child device 12 in the third embodiment of the obstacle notification system 1 in FIG. 1. The same reference numerals are assigned to the parts common to those of the parent device 11 and the child device 12 in FIG. 4, and the description thereof will be omitted as appropriate.

The parent device 11 in FIG. 6 has a data receiving unit 41, a DSP 43, and an audio output unit 44. The child device 12 in FIG. 6 has an obstacle ranging sensor 61, a data transmitting unit 62, and a child device tracking unit 64. Therefore, the parent device 11 in FIG. 6 is common to the parent device 11 in FIG. 4 in that it has the data receiving unit 41, the DSP 43, and the audio output unit 44. The child device 12 in FIG. 6 is common to the child device 12 in FIG. 4 in that it has the obstacle ranging sensor 61 and the data transmitting unit 62.

However, the parent device 11 in FIG. 6 is different from the parent device 11 in FIG. 4 in that it does not have the child device tracking unit 42. The child device 12 in FIG. 6 is different from the child device 12 in FIG. 4 in that a child device tracking unit 64 is newly provided.

In FIG. 6, the child device tracking unit 64 tracks the child device 12, and measures the distance and direction (parent device-child device distance and direction) of the child device 12 with respect to the parent device 11 and the attitude (parent device-child device attitude) of the child device 12 with respect to to the parent device 11. The child device tracking unit 64 supplies the parent device-child device distance and direction and the parent device-child device attitude obtained by the measurement to the DSP 43 of the parent device 11 via the data transmitting unit 62 and the data receiving unit 41 as the information indicating the relative positional relationship and attitude between the parent device 11 and the child device 12.

Instead of obtaining the parent device-child device distance and direction and the parent device-child device attitude from the child device tracking unit 42 in FIG. 4, the DSP 43 obtains the parent device-child device distance and direction and the parent device-child device attitude from the child device tracking unit 64 in FIG. 6 as the information indicating the relative positional relationship and attitude between the parent device 11 and the child device 12.

As a result, in the obstacle position calculation processing, as in the case in FIG. 4, the DPS 43 calculates the distance and direction (parent device-obstacle vector V) of the measurement point C of the obstacle 22 with respect to the three-dimensional position of the parent device 11 based on the child device-obstacle distance obtained from the obstacle ranging sensor 61 and the parent device-child device distance and direction and the parent device-child device attitude from the child device tracking unit 64.

According to the third embodiment of the obstacle notification system 1 described above, the parent device 11 can be made smaller than the first embodiment, and the burden on the head of the user 21 can be reduced. Therefore, the distance and direction from the head of the user 21 can be measured even for an obstacle (the measurement point C) present at a distance where the same cannot be measured from the head of the user 21. Regardless of the position of the child device 12 having the range-finding function with respect to the head of the user 21, since a notification sound for allowing the user 21 to perceive the distance and direction of the obstacle (the measurement point C) based on the head of the user 21 at the position of the sound image is always presented to the user 21, the user 21 can reliably and stably perceive obstacles present in the surroundings.

### Fourth Embodiment of Obstacle Notification System 1

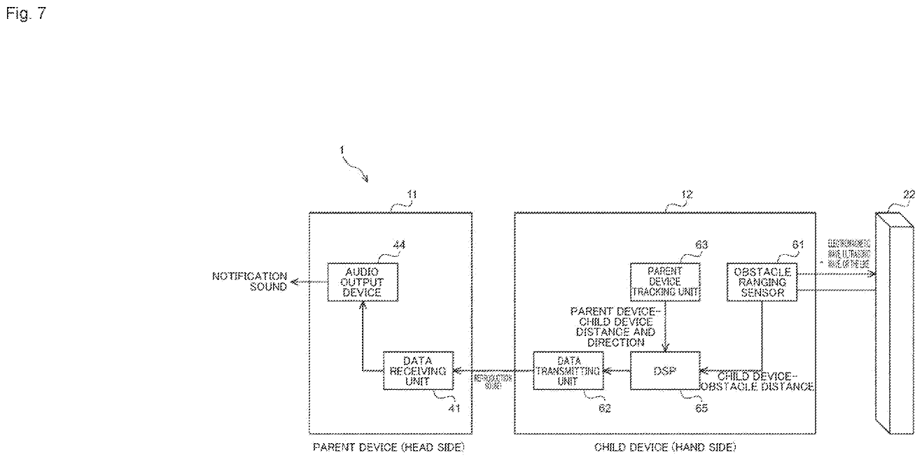

FIG. 7 is a block diagram illustrating the internal configuration of the parent device 11 and the child device 12 in the fourth embodiment of the obstacle notification system 1 in FIG. 1. The same reference numerals are assigned to the parts common to those of the parent device 11 and the child device 12 in FIG. 5, and the description thereof will be omitted as appropriate.

The parent device 11 in FIG. 7 has a data receiving unit 41 and an audio output unit 44. The child device 12 in FIG. 7 has an obstacle ranging sensor 61, a data transmitting unit 62, a parent device tracking unit 63 and a DSP 65. Therefore, the parent device 11 in FIG. 7 is common to the parent device 11 in FIG. 5 in that it has the data receiving unit 41 and the audio output unit 44. The child device 12 in FIG. 7 is common to the child device 12 in FIG. 5 in that it has the obstacle ranging sensor 61, the data transmitting unit 62, and the parent device tracking unit 63. However, the parent device 11 in FIG. 7 is different from the parent device 11 in FIG. 5 in that it does not have the DSP 43. The child device 12 in FIG. 7 is different from the child device 12 in FIG. 5 in that a DSP 65 is newly provided.

In FIG. 7, the DSP 65 obtains the parent device-child device distance and direction and the parent device-child device attitude from the parent device tracking unit 63 as the information indicating the relative positional relationship and attitude between the parent device 11 and the child device 12. The DSP 65 obtains the child device-obstacle distance from the obstacle ranging sensor 61. Similarly to the DSP 43 in FIG. 5 (FIG. 4), the DSP 65 performs sound image localization calculation including the obstacle position calculation processing and the notification sound generation processing based on the child device-obstacle distance from the obstacle ranging sensor 61 and the parent device-child device distance and direction and the parent device-child device attitude from the parent device tracking unit 63 to generate the right (right-ear) notification sound and the left (left-ear) notification sound. The DSP 65 transmits the generated notification sounds to the parent device 11 via the data transmitting unit 62 and the data receiving unit 41 and supplies the same to the audio output unit 44.

According to the fourth embodiment of the obstacle notification system 1 described above, the parent device 11 can be made smaller than the first embodiment, and the burden on the head of the user 21 can be reduced. Since the notification sound is only transmitted to the parent device 11 as a reproduction sound signal, an existing audio output device such as Bluetooth (registered trademark) earphones can be used as the parent device 11. Therefore, the distance and direction from the head of the user 21 can be measured even for an obstacle (the measurement point C) present at a distance where the same cannot be measured from the head of the user 21. Regardless of the position of the child device 12 having the range-finding function with respect to the head of the user 21, since a notification sound for allowing the user 21 to perceive the distance and direction of the obstacle (the measurement point C) based on the head of the user 21 at the position of the sound image is always presented to the user 21, the user 21 can reliably and stably perceive obstacles present in the surroundings.

### Modification Example of Obstacle Notification System 1

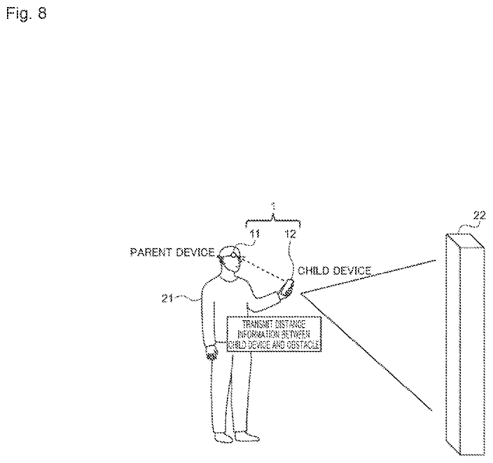

FIG. 8 is a configuration diagram showing a modification of the obstacle notification system 1 in FIG. 1. The same reference numerals are assigned to the parts common to those of the obstacle notification system 1 in FIG. 1, and the description thereof will be omitted as appropriate.

In the modification example of the obstacle notification system 1 in FIG. 8, using the child device 12 as a reference, distances to a plurality of measurement points on an obstacle (for example, the surface region of the obstacle 22) present within a predetermined angular range with respect to a specific measurement direction.

Since the configuration example of the parent device 11 and the child device 12 in the modification example of the obstacle notification system 1 in FIG. 8 is the same as the obstacle notification system 1 in FIG. 1, a modification example will be described using the configuration example in FIG. 4. The description of the portions where the same processing as in the obstacle notification system 1 in FIG. 1 is performed will be omitted.

In FIG. 8, a ranging sensor capable of multi-directional ranging around a specific measurement direction with respect to the child device 12 is used as the obstacle ranging sensor 61. In this modification example, for example, an optical depth sensor is used as the obstacle ranging sensor 61. However, the obstacle ranging sensor 61 may be a Lidar, radar, stereo camera, or the like.

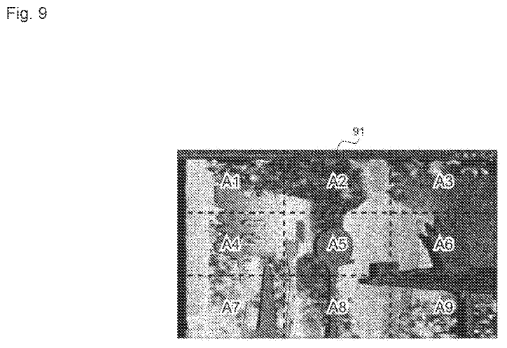

FIG. 9 is a diagram illustrating a depth image obtained by the obstacle ranging sensor 61.

In FIG. 9, the depth image 91 is an image in which the pixel value of each pixel corresponds to the measured distance to the obstacle (object).

When the obstacle ranging sensor 61 supplies the information of the depth image 91 obtained by measurement as it is to the DSP 43 via the data transmitting unit 62 and the data receiving unit 41, the amount of data transmission becomes enormous. In the case of wireless transmission from child device 12 to parent device 11, it is necessary to use a large transmission band.

Therefore, the obstacle ranging sensor 61 divides the range (measurement area) of the depth image 91 obtained by ranging into a plurality of divided areas A1 to A9. The obstacle ranging sensor 61 obtains the average value, the maximum value, or the minimum value of the pixel values (distances) of the divided areas A1 to A9 as a representative value. However, the method of calculating the representative value is not limited to this. The obstacle ranging sensor 61 associates the central direction of each of the divided areas A1 to A9 with the distance indicated by the representative value of the corresponding divided area. The obstacle ranging sensor 61 supplies the distance and direction of those measurement points to the DSP 43. In this way, the amount of data transmission is reduced. In the obstacle notification system 1 in FIG. 1, the three-dimensional positions of a plurality of (nine) measurement points (the measurement points in different measurement directions) corresponding to one measurement point C are specified by the DSP 43.

In the obstacle notification system 1 in FIG. 1, the direction (measurement direction) of the measurement point C is determined in advance with respect to the child device 12 (the child device coordinate system). When the DSP 43 has grasped the measurement direction in advance, the DSP 43 can obtain only the child device-obstacle distance from the obstacle ranging sensor 61, and calculate the three-dimensional position of the measurement point C in the child device coordinate system. In this modification example in FIG. 8, by grasping the directions of a plurality of measurement points with respect to the child device 12 in advance, the DSP 43 can obtain only the child device-obstacle distance with respect to each measurement point from the obstacle ranging sensor 61 and calculate the three-dimensional position of each measurement point in the child device coordinate system.

The DSP 34 performs the obstacle position calculation processing on a plurality of measurement points in the same manner as in the case of the obstacle notification system 1 in FIG. 1 and calculates the distance and direction of each measurement point with respect to the three-dimensional position of the parent device 11 to specify the three-dimensional position of each measurement point in the parent device coordinate system.

The DSP 34 performs the notification sound generation processing on a plurality of measurement points in the same manner as in the case of the obstacle notification system 1 in FIG. 1 to generate a right notification sound and a left notification sound when an original sound is emitted using the three-dimensional positions of a plurality of measurement points as the position of a virtual sound source. At this time, a plurality of notification sounds corresponding to a plurality of measurement points are generated as the right and left notification sounds. The DSP 34 integrates a plurality of right notification sounds into one notification sound by addition or the like, and integrates a plurality of left notification sounds into one notification sound by addition or the like. The notification sound may be generated such that the three-dimensional position of the sound source is different from the plurality of measurement points, and the three-dimensional positions of the plurality of measurement points are set as the reflection positions of the original sound from the sound source.

The DSP 34 presents the notification sound to the user 21 by supplying the generated notification sound to the audio output unit 44.

According to the modification example of the obstacle notification system 1 in FIG. 8, detailed information such as the rough shape and area of the obstacle is presented to the user 21 by stereophonic sound. Therefore, the distance and direction from the head of the user 21 can be measured even for an obstacle (the measurement point C) present at a distance where the same cannot be measured from the head of the user 21. Regardless of the position of the child device 12 having the range-finding function with respect to the head of the user 21, since a notification sound for allowing the user 21 to perceive the distance and direction of the obstacle (the measurement point C) based on the head of the user 21 at the position of the sound image is always presented to the user 21, the user 21 can reliably and stably perceive obstacles present in the surroundings.

The present technology be used as a technology for detecting the presence of an object in a blind spot since the user having received the notification can perceive the presence of an object that the user cannot directly see. The present technology is also effective when a ranging sensor is installed in a vehicle such as an automobile. In this case, a ranging sensor (the child device having a range-finding function) is installed on the exterior part of the automobile, and the parent device 11 is arranged on the user's body such as the head or in the vicinity of the user. A speaker in the vehicle may be used to notify the user by the notification sound.

<Program>

The series of processing in the obstacle notification system 1, the parent device 11, or the child device 12 described above can be executed by hardware or by software. In a case where the series of processing is executed by software, a program that constitutes the software is installed on a computer. Here, the computer includes a computer embedded in dedicated hardware or, for example, a general-purpose personal computer capable of executing various functions by installing various programs.

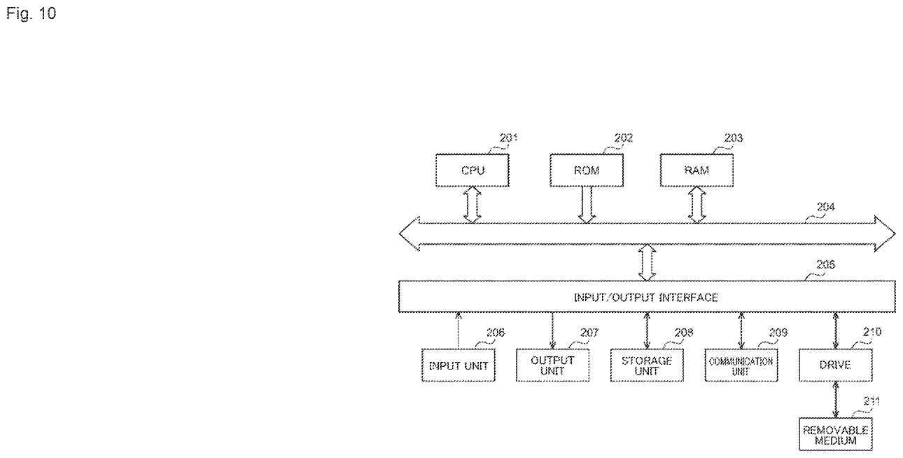

FIG. 10 is a block diagram showing a hardware configuration example of a computer when the computer executes the series of processing executed by the obstacle notification system 1, the parent device 11, or the child device 12 by means of a program.

In the computer, a central processing unit (CPU) 201, a read only memory (ROM) 202, and a random access memory (RAM) 203 are connected to each other by a bus 204.

An input/output interface 205 is further connected to the bus 204. An input unit 206, an output unit 207, a storage unit 208, a communication unit 209, and a drive 210 are connected to the input/output interface 205.

The input unit 206 is constituted by a keyboard, a mouse, a microphone, or the like. The output unit 207 is constituted of a display, a speaker, or the like. The storage unit 208 is a hard disk, non-volatile memory, or the like. The communication unit 209 is a network interface or the like. The drive 210 drives a removable medium 211 such as a magnetic disk, an optical disc, a magneto-optical disk, or a semiconductor memory.

In the computer configured as described above, for example, the CPU 201 loads a program stored in the storage unit 208 into the RAM 203 via the input/output interface 205 and the bus 204 and executes the program to perform the series of processing described above.

The program executed by the computer (the CPU 201) can be recorded on, for example, the removable medium 211 serving as a package medium for supply. The program can be supplied via a wired or wireless transmission medium such as a local area network, the Internet, or digital satellite broadcasting.

In the computer, by mounting the removable medium 211 on the drive 210, it is possible to install the program in the storage unit 208 via the input/output interface 205. The program can be received by the communication unit 209 via a wired or wireless transfer medium and can be installed in the storage unit 208. In addition, this program may be installed in advance in the ROM 202 or the storage unit 208.

Note that the program executed by a computer may be a program that performs processing chronologically in the order described in the present specification or may be a program that performs processing in parallel or at a necessary timing such as a called time.

The present technique can be also configured as follows:

(1)

An information processing device including:

a processing unit that calculates a distance and a direction of a measurement point with respect to a first position in a first coordinate system based on a relative distance and direction in the first coordinate system or a second coordinate system between the first position whose coordinates in the first coordinate system are determined and a second position whose coordinates in the second coordinate system are determined, the second position being separated from the first position,

a relative attitude between the first coordinate system and the second coordinate system, and

a distance to the measurement point present in a predetermined measurement direction in the second coordinate system, measured from the second position, and generates a notification signal to be presented to a user based on at least one of the distance and the direction of the measurement point with respect to the first position.

(2)

The information processing device according to (1), wherein the first position and the second position are positions set on individual objects.

(3)

The information processing device according to (2), wherein the first position is a position of the user's head.

(4)

The information processing device according to (2) or (3), wherein the second position is a position other than the user's head.

(5)

The information processing device according to any one of (2) to (4), wherein the first coordinate system is a coordinate system set in a first device arranged on the user's head.

(6)

The information processing device according to (5), wherein

the first position is a position of the first device.

(7)

The information processing device according to any one of (2) to (6), wherein the second coordinate system is a coordinate system set in a second device arranged at a position other than the user's head.

(8)

The information processing device according to (7), wherein the second device is a device whose arrangement position is not determined to be a fixed position.

(9)

The information processing device according to (7) or (8), wherein the second position is a position of the second device

(10)

The information processing device according to any one of (7) to (9), wherein the second device has a ranging sensor that measures the distance to the measurement point.

(11)

The information processing device according to any one of (1) to (10), wherein the notification signal is a sound signal for presenting a notification sound to the user.

(12)

The information processing device according to (11), wherein the sound signal is a stereo sound signal composed of a right sound signal and a left sound signal.

(13)

The information processing device according to (11) or (12), wherein the processing unit generates the notification signal that allows the user to perceive the position of the measurement point as a position of a sound image.

(14)

The information processing device according to any one of (11) to (13), wherein the processing unit assumes that an original sound that is a source of the notification sound is emitted or reflected at the position of the measurement point, and generates a sound signal indicating a sound when the original sound reaches the user's head as the notification signal.

(15)

The information processing device according to (14), wherein the processing unit generates sound signals representing sounds when the original sound reaches the user's right ear and left ear, respectively, as the notification signal for the right ear and the notification signal for the left ear.

(16)

The information processing device according to (14) or (15), wherein the processing unit generates the notification signal by performing at least one of processing of convolving the original sound with a head-related transfer function according to a propagation path until the original sound reaches the head, processing of delaying the original sound according to a length of the propagation path, and processing of attenuating a volume according to the length of the propagation path.

(17)

The information processing device according to any one of (1) to (16), wherein there are a plurality of measurement points with different measurement directions.

(18)

The information processing device according to (17), wherein the processing unit assumes that the original sound that is the source of the notification signal that presents a sound to the user is emitted or reflected at the positions of the plurality of measurement points, and generates a sound signal indicating the sound when the original sound reaches the user's head as the notification signal.

(19)

An information processing method for causing a processing unit of an information processing device to execute:

calculating a distance and a direction of a measurement point with respect to a first position in a first coordinate system based on a relative distance and direction in the first coordinate system or a second coordinate system between the first position whose coordinates in the first coordinate system are determined and a second position whose coordinates in the second coordinate system are determined, the second position being separated from the first position, a relative attitude between the first coordinate system and the second coordinate system, and a distance to the measurement point present in a predetermined measurement direction in the second coordinate system, measured from the second position, and generating a notification signal to be presented to a user based on at least one of the distance and the direction of the measurement point with respect to the first position.

(20)

A program for causing a computer to function as:

a processing unit that calculates a distance and a direction of a measurement point with respect to a first position in a first coordinate system based on a relative distance and direction in the first coordinate system or a second coordinate system between the first position whose coordinates in the first coordinate system are determined and a second position whose coordinates in the second coordinate system are determined, the second position being separated from the first position,

a relative attitude between the first coordinate system and the second coordinate system, and

a distance to the measurement point present in a predetermined measurement direction in the second coordinate system, measured from the second position, and generates a notification signal to be presented to a user based on at least one of the distance and the direction of the measurement point with respect to the first position.

### REFERENCE SIGNS LIST

1 Obstacle notification system
11 Parent device
12 Child device
41 Data receiving unit
42 Child device tracking unit
43 DSP
44 Audio output unit
61 Obstacle ranging sensor
62 Data transmitting unit

1. An information processing device comprising:
a processing unit that calculates a distance and a direction of a measurement point with respect to a first position in a first coordinate system based on a relative distance and direction in the first coordinate system or a second coordinate system between the first position whose coordinates in the first coordinate system are determined and a second position whose coordinates in the second coordinate system are determined, the second position being separated from the first position,
a relative attitude between the first coordinate system and the second coordinate system, and
a distance to the measurement point present in a predetermined measurement direction in the second coordinate system, measured from the second position, and generates a notification signal to be presented to a user based on at least one of the distance and the direction of the measurement point with respect to the first position.

2. The information processing device according to claim 1, wherein
the first position and the second position are positions set on individual objects.

3. The information processing device according to claim 2, wherein
the first position is a position of the user's head.

4. The information processing device according to claim 2, wherein
the second position is a position other than the user's head.

5. The information processing device according to claim 2, wherein
the first coordinate system is a coordinate system set in a first device arranged on the user's head.

6. The information processing device according to claim 5, wherein
the first position is a position of the first device.

7. The information processing device according to claim 2, wherein
the second coordinate system is a coordinate system set in a second device arranged at a position other than the user's head.

8. The information processing device according to claim 7, wherein
the second device is a device whose arrangement position is not determined to be a fixed position.

9. The information processing device according to claim 7, wherein
the second position is a position of the second device

10. The information processing device according to claim 7, wherein
the second device has a ranging sensor that measures the distance to the measurement point.

11. The information processing device according to claim 1, wherein
the notification signal is a sound signal for presenting a notification sound to the user.

12. The information processing device according to claim 11, wherein
the sound signal is a stereo sound signal composed of a right sound signal and a left sound signal.

13. The information processing device according to claim 11, wherein
the processing unit generates the notification signal that allows the user to perceive the position of the measurement point as a position of a sound image.

14. The information processing device according to claim 11, wherein
the processing unit assumes that an original sound that is a source of the notification sound is emitted or reflected at the position of the measurement point, and generates a sound signal indicating a sound when the original sound reaches the user's head as the notification signal.

15. The information processing device according to claim 14, wherein
the processing unit generates sound signals representing sounds when the original sound reaches the user's right ear and left ear, respectively, as the notification signal for the right ear and the notification signal for the left ear.

16. The information processing device according to claim 14, wherein
the processing unit generates the notification signal by performing at least one of processing of convolving the original sound with a head-related transfer function according to a propagation path until the original sound reaches the head, processing of delaying the original sound according to a length of the propagation path, and processing of attenuating a volume according to the length of the propagation path.

17. The information processing device according to claim 1, wherein there are a plurality of measurement points with different measurement directions.

18. The information processing device according to claim 17, wherein the processing unit assumes that an original sound that is a source of the notification signal that presents a sound to the user is emitted or reflected at positions of the plurality of measurement points, and generates a sound signal indicating a sound when the original sound reaches the user's head as the notification signal.

19. An information processing method for causing a processing unit of an information processing device to execute:
calculating a distance and a direction of a measurement point with respect to a first position in a first coordinate system based on a relative distance and direction in the first coordinate system or a second coordinate system between the first position whose coordinates in the first coordinate system are determined and a second position whose coordinates in the second coordinate system are determined, the second position being separated from the first position, a relative attitude between the first coordinate system and the second coordinate system, and a distance to the measurement point present in a predetermined measurement direction in the second coordinate system, measured from the second position, and generating a notification signal to be presented to a user based on at least one of the distance and the direction of the measurement point with respect to the first position.

20. A program for causing a computer to function as: a processing unit that calculates a distance and a direction of a measurement point with respect to a first position in a first coordinate system based on a relative distance and direction in the first coordinate system or a second coordinate system between the first position whose coordinates in the first coordinate system are determined and a second position whose coordinates in the second coordinate system are determined, the second position being separated from the first position,
a relative attitude between the first coordinate system and the second coordinate system, and
a distance to the measurement point present in a predetermined measurement direction in the second coordinate system, measured from the second position, and generates a notification signal to be presented to a user based on at least one of the distance and the direction of the measurement point with respect to the first position.
